(19) United States
(12) Patent Application Publication
(10) Pub. No.: US 2017/0050040 A1
(43) Pub. Date: Feb. 23, 2017

(54) METHOD AND MAGNETIC CATHETER FOR MAGNETIC NANOPARTICLE TREATMENT OF THE PROSTATE

(71) Applicant: The Trustees of Dartmouth College, Hanover, NH (US)

(72) Inventor: Stuart B. Trembly, Hanover, NH (US)

(21) Appl. No.: 15/307,776

(22) PCT Filed: Apr. 28, 2015
(86) PCT No.: PCT/US15/27891

§ 371 Date: Oct. 28, 2016

Related U.S. Application Data
(60) Provisional application No. 61/985,164, filed on Apr. 28, 2014.

(51) Int. Cl. A61N 2/06; A61M 25/10

(52) U.S. Cl. CPC A61N 2/06; A61M 25/10

(57) ABSTRACT

A magnetic catheter has a flexible tube having a lumen communicating with a balloon, the balloon located near a distal end of the tube; and a nonuniform magnetic portion located near, but proximal to, the balloon. The tube is adapted to insertion into bodily orifices like a male urethra, the lumen extending from balloon to proximal end and configured such that injection of fluid into the first lumen can inflate the balloon. The nonuniform magnetic portion has a sequence of magnetically permeable objects, or a sequence of permanent magnets, to provide magnetic field gradients to the prostate, or in alternative embodiment the pancreas. The catheter is used to treat the organ by applying a nonuniform magnetic field to the organ while magnetic nanoparticles are injected into the patient where they concentrate in the nonuniform magnetic field. An AC magnetic field is applied to the magnetic nanoparticles to heat the organ.

### RELATED APPLICATION

The present document claims priority to U.S. Provisional Patent Application 61/985,164 filed 28 Apr. 2014.

### GOVERNMENT INTEREST

Work described herein has been sponsored by the National Institutes of Health, National Cancer Institute grant number 5U54CA151662. The government has certain rights in the invention.

### BACKGROUND

Benign prostatic hyperplasia (BPH) affects more than half of all men older than 50 years and 90% of men in their 80s, producing symptoms of frequent urination, weak urinary stream, incomplete bladder emptying, and others. Men who do not respond to medication or who experience undesirable side effects may elect minimally invasive treatment options, such as transurethral needle ablation (TUNA) or transurethral microwave therapy (TUMT). Invasive treatment options include prostatic arterial embolization, transurethral resection (TURP) surgery or various laser procedures. In TURP, a transurethral electrified loop resects prostate tissue while simultaneously cauterizing. State of the art for prostatic arterial embolism is described in Cardiovasc Intervent Radiol (2014) 37:1602-1605, DOI 10.1007/s00270-014-0908-z, The “PErFecTED Technique”: Proximal Embolization First, Then Embolize Distal for Benign Prostatic Hyperplasia, Francisco C. Carnevale, et al. Historically, surgery such as TURP has provided better results than minimally invasive surgery: a systematic review showed that urinary flow increased 1.7 times after TUMT and 2.2 times after TURP, Hoffman, R. M., R. MacDonald, M. Monga and T. J. Wilt, 2004. Transurethral Microwave Thermotherapy Vs Transurethral Resection For Treating Benign Prostatic Hyperplasia: A Systematic Review. BJU International 94:1031-1036. However, TURP requires spinal or general anesthesia, entails a hospital stay, and may produce significant intraoperative and postoperative bleeding, possibly requiring a blood transfusion. By contrast, the minimally-invasive therapies are office-based, require minimal anesthesia, and produce less morbidity.

Prostate cancer is also quite common among elderly men in the United States, and is sometimes fatal. Current prostate cancer treatments leave much to be desired, since surgery often damages the nerves required for an erection and is therefore unpopular among men. There is need for an improved prostate cancer treatment, as well as BPH treatment.

Hyperthermal therapy has been used for a variety of purposes over many years. While heat at some temperatures can dilate blood vessels and help heal wounds, as well as help treat bacterial infections, and greater heat can kill tumors, heat can also kill necessary tissues and cause other problems for patients.

It is believed that more precise application of heat to specific tissues will permit greater use of hyperthermal therapies on deeper tissues and organs than has been common in the past. As a way of providing heat delivery to deep tissues, it has been proposed to use the interaction of time-varying, or AC, magnetic fields with magnetic nanoparticles to deliver heat to tissues, as described in U.S. Pat. No. 8,565,892 to Nayfach-Battilana ('892) and U.S. patent application Ser. No. 10/360,578. The AC magnetic field provided for heating is typically at a frequency between 30 kHz and 200 kHz.

It has been previously reported, for example by Polyak et al (“High field gradient targeting of magnetic nanoparticle-loaded endothelial cells to the surfaces of steel stents”, 2008), that a high magnetic field gradient at the ends of a stent may attract magnetic nanoparticles to the vicinity of ends of the steel stent. The stent had been threaded through an incision into a blood vessel and implanted in an artery at a particular site in a rat. Polyak does not disclose heating using those nanoparticles, or magnetic material inserted through a natural bodily orifice or through a duct other than a blood vessel. In particular, Polyak does not disclose a nonuniform magnetic material. Further, stents are nonremovable and are typically placed in a blood vessel. It may, however, be undesirable to place a nonremovable stent in an organ having a tumor requiring treatment because presence of a magnetically permeable stent can disrupt some imaging modalities, including nuclear magnetic resonance imaging (nMRI), that are often used for observing tumor growth to monitor treatment effectiveness.

When magnetic nanoparticles are used to provide localized, deep, heating for thermal therapies, heating can be directed to particular tissues by either or both of positioning those particles in or near the tissue to be treated, or focusing the time-varying fields in or near the tissue to be treated. The approach taken in '892 to positioning particles in tissues is based on a tissue-selective coating that traps nanoparticles at the tissue to be treated.

It has been proposed that prostate cancer be treated using magnetic nanoparticles, Int. J. Hyperthermia, December 2010; 26(8): 790-795 Magnetic nanoparticle hyperthermia for prostate cancer Manfred Johannsen, et al. Johannsen proposes directly injecting nanoparticles into the organ, or into tumor in the organ, followed by using an externally-applied time-varying, or AC, magnetic field to heat the particles; Johannsen combined nanoparticle hyperthermia with radiation treatments in some patients of his study.

### SUMMARY

In an embodiment, a magnetic catheter has a flexible tube having at least a first lumen communicating with a balloon, the balloon located near a distal end of the tube; a nonuniform magnetic portion located near, but proximal to, the balloon. The tube adapted to insertion into a male urethra, the first lumen extending from the balloon to a proximal end and configured such that injection of fluid into the first lumen can inflate the balloon. The nonuniform magnetic portion has either a sequence of magnetically permeable objects, or a sequence of permanent magnets. The catheter is used to treat the prostate by applying a nonuniform magnetic field to the prostate; magnetic nanoparticles are then injected into the patient where they concentrate in the nonuniform magnetic field. A time-varying, or AC, magnetic field is then applied to the magnetic nanoparticles to heat the prostate.

In an embodiment, a magnetic catheter includes a flexible shaft, a nonuniform magnetic portion comprising nonuniform magnetic elements selected from a nonuniform permanent magnet portion and a nonuniform magnetically permeable portion. In a particular embodiment the flexible shaft includes a flexible tube having at least a first lumen, the first lumen communicating with a balloon, the balloon located near a distal end of the flexible tube; wherein the nonuniform magnetic portion is located near, but proximal to, the balloon; and wherein the catheters is adapted to having the distal end inserted through a penile urethra into a urinary bladder of a male patient, the first lumen extending from the distal end to a proximal end and configured such that injection of fluid into a proximal end of the first lumen can inflate the balloon. In a different particular embodiment, the catheter is adapted to be inserted through a lumen of an endoscope by having sufficient length, flexibility and small diameter to fit through the lumen of the endoscope.

In another embodiment, system has a magnetic catheter having a flexible shaft, and a nonuniform magnetic portion comprising nonuniform magnetic elements selected from a nonuniform permanent magnet portion and a nonuniform magnetically permeable portion; apparatus for administering magnetic nanoparticles to a subject; and apparatus for applying an alternating magnetic field to the subject.

In another embodiment, a method of treating an organ of a patient includes applying a nonuniform magnetic field to the organ by a method including inserting a catheter having a nonuniform magnetic portion into a lumen of a duct or vessel of the patient located within the organ; injecting magnetic nanoparticles into the patient; allowing the magnetic nanoparticles to concentrate in the nonuniform magnetic field within the organ; removing the catheter; and applying a time-varying magnetic field to the magnetic nanoparticles.

In an embodiment, a system for treatment of the prostate has a magnetic catheter comprising a nonuniform magnetic portion located near a distal end of the catheter, the catheter adapted to be inserted through a penile urethra into a urinary bladder of a male patient, and a suspension of magnetic nanoparticles contained within an apparatus adapted to inject the nanoparticles into the patient. The system also has an apparatus for providing an alternating magnetic field to a prostate of the patient and adapted to heating any magnetic nanoparticles within the prostate.

### BRIEF DESCRIPTION OF THE DRAWINGS

FIG. 1 is a schematic diagram of an embodiment of a magnetic catheter.

FIG. 2 is a diagram of an embodiment of a magnetic portion of the magnetic catheter of FIG. 1

FIG. 2A is a diagram of an alternative embodiment of the magnetic portion of the catheter of FIG. 1.

FIG. 3 is a diagram of an alternative embodiment of a magnetic portion of the magnetic catheter of FIG. 1

FIG. 4 is a schematic system diagram of a patient illustrating the magnetic catheter in use in a patient.

FIG. 5 is a flowchart illustrating use of the magnetic catheter with nanoparticles to destroy a tumor of the prostate or to treat benign prostate hypertrophy.

FIG. 6 is a schematic illustration of an alternative embodiment applied to thermotherapy of the pancreas.

FIG. 7 is a schematic illustration of an alternative embodiment applied to thermotherapy of a lesion adjacent to the esophagus.

FIG. 8 is a schematic cross-sectional illustration of a magnetic endoscope head such as may be used for magnetic nanoparticle thermotherapy of tissue located near a subject's esophagus or stomach.

FIG. 9 is a schematic cross-sectional illustration of a non-magnetic water-cooled catheter configured to be placed in a body lumen, such as the urethra or esophagus, to protect lumen lining during magnetic particle thermotherapy.

FIG. 9A is an illustration of a spacer such as the spacer of the catheter of FIG. 9.

### DETAILED DESCRIPTION OF THE EMBODIMENTS

This invention seeks to improve upon the minimally-invasive treatments including prostatic arterial embolization by eliminating the need to inject the embolizing agent in the artery immediately upstream of tissue to be destroyed, and upon TUMT and TUNA by eliminating the need to place an energy source within the urethra with which to heat the surrounding prostate. The practical result will be a transurethral catheter of smaller diameter, which will produce less discomfort and morbidity and, consequently, greater patient acceptance. This invention will reduce the diameter of the treatment catheter by eliminating the lumen for a microwave antenna and its feedline or wires to carry radio-frequency current. Catheter lumens for a urothelium cooling system and for urethral thermometry will be incorporated for the sake of their safety functions.

The invention will eliminate other known disadvantages of microwave antennas in heating the prostate, namely, the back-heating along the feedline, the effect of antenna design on heating profile, and the small fraction of total power that reaches the prostate. The inventor is familiar from his own past research with the characteristic back-heating along the feedline of a microwave antenna in the prostate (Trembly, B. S., P. J. Hoopes, K. L. Moodie and A. S. Dvinsky 1999. Microwave Antenna Array For Prostate Hyperthermia. Proceedings of the International Symposium on Biomedical Optics, International Society for Optical Engineering, San Jose, Calif., 23-29 Jan. 1999). The proposed system will inherently deposit energy only in periurethral tissue; consequently, the variable and misdirected power deposition noted above will be eliminated. In comparison to TUNA, the proposed system inherently will heat at a constant rate; the heating rate will not vary with temperature-dependent changes in the electrical conductivity of tissue, as is the case with any radio-frequency system (Ryan T P, Interstitial microwave transition form hyperthermia to ablation: Historical perspectives and current trends in thermal therapy, International Journal of Hyperthermia, 26(5), 415-433, August, 2010.). In addition to treating benign prostatic hyperplasia, the invention can treat prostate cancer, as well as cancer at other anatomical sites.

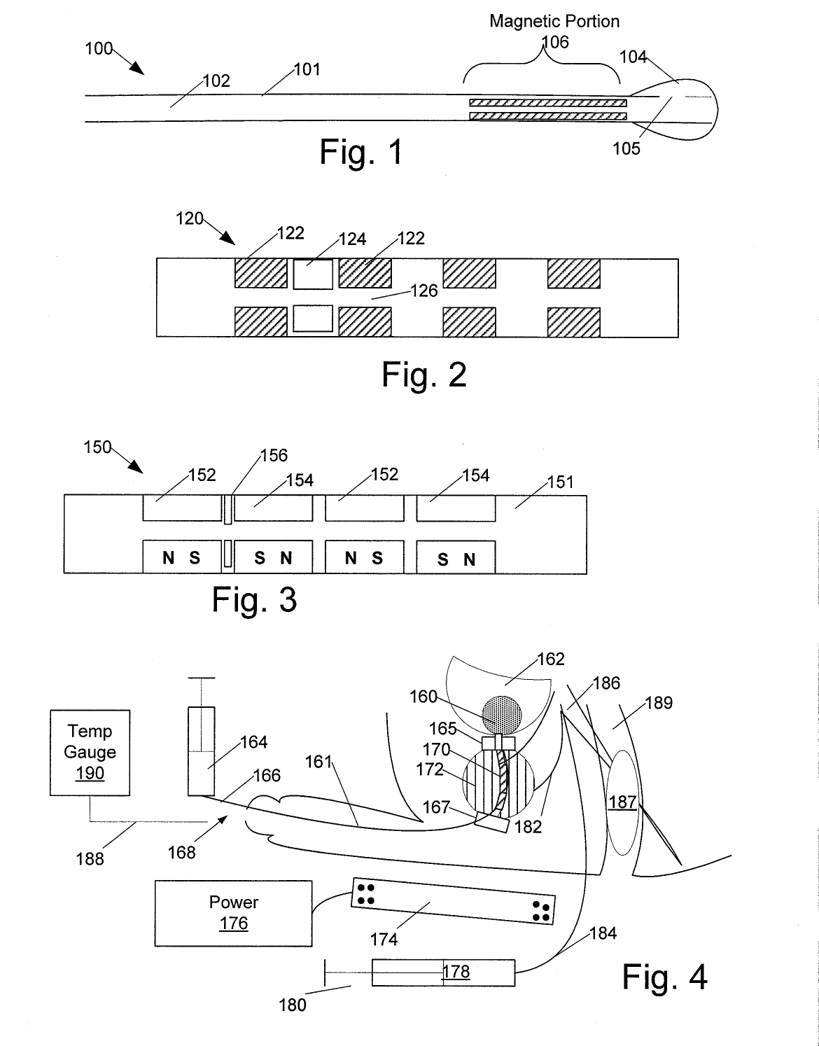

A magnetic catheter 100 (FIG. 1) has a flexible tubular portion 101 with at least one lumen 102 similar to that of urinary catheters in common use. Some embodiments of the catheter have a balloon portion 104 that communicates, for example through a hole 105 in a wall of tube, with lumen 102. The balloon portion is located at or near a distal end of the catheter. The catheter has a magnetic portion 106 located near the distal end, but proximal to balloon 104, the magnetic portion 106 occupies a length of the catheter preferably approximately equal to a length of urethra that lies within the prostate and defines a length of a treatment zone within the prostate, so as to exclude the bladder or internal sphincter 165 (formed of muscle near the junction of bladder and urethra above the prostate) and external sphincter 167 (formed of skeletal muscles of the perineum beneath the prostate). In some embodiments it is desirable to avoid excessive damage to the sphincters 165, 167 to avoid leaving patients incontinent, these sphincters are protected by sizing and positioning the magnetic portion such that no strong magnetic field gradients are present in those sphincters, and therefore the magnetic nanoparticles do not accumulate in the sphincters to the extent that they accumulate in the prostate. The magnetic portion is configured such that it ends at or near a lower border of the prostate when positioned within the prostate such that it does not extend proximally to the external urethral sphincter. For purposes of this document, nanoparticles are particles having size between ten nanometers and 1 micron. Magnetic nanoparticles are nanoparticles consisting of, or having a core of, a ferromagnetic material such as an iron oxide. Magnetic nanoparticles may or may not contain other materials as well, including coatings of biocompatible materials, such coatings may include one or more of dispersing agents, tissue selective or nonselective binding agents that serve to attach the particles to cells, and penetration enhancing agents that permit the particles to pass through cell membranes into cells.

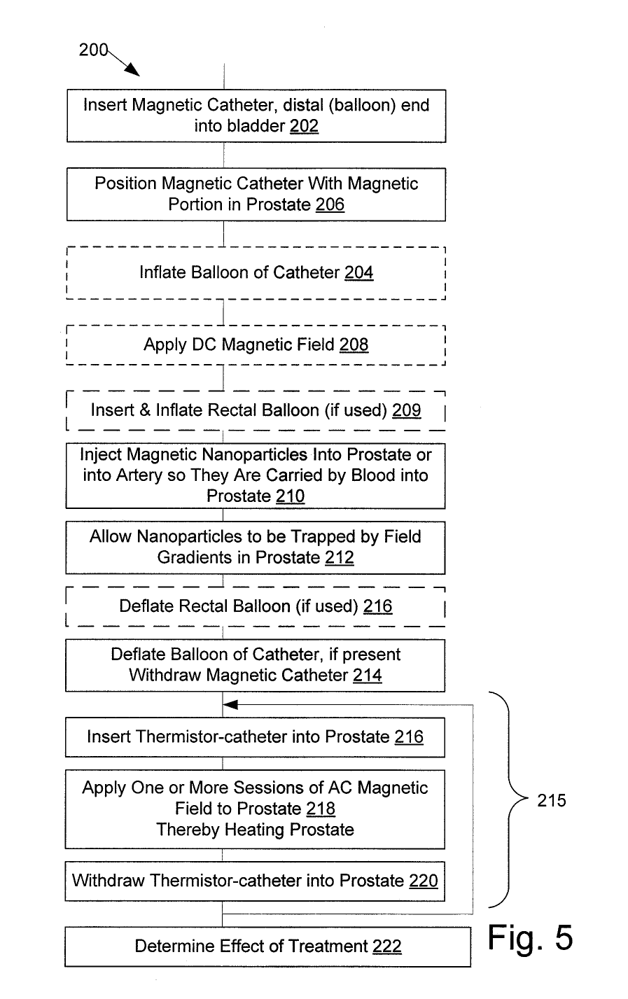

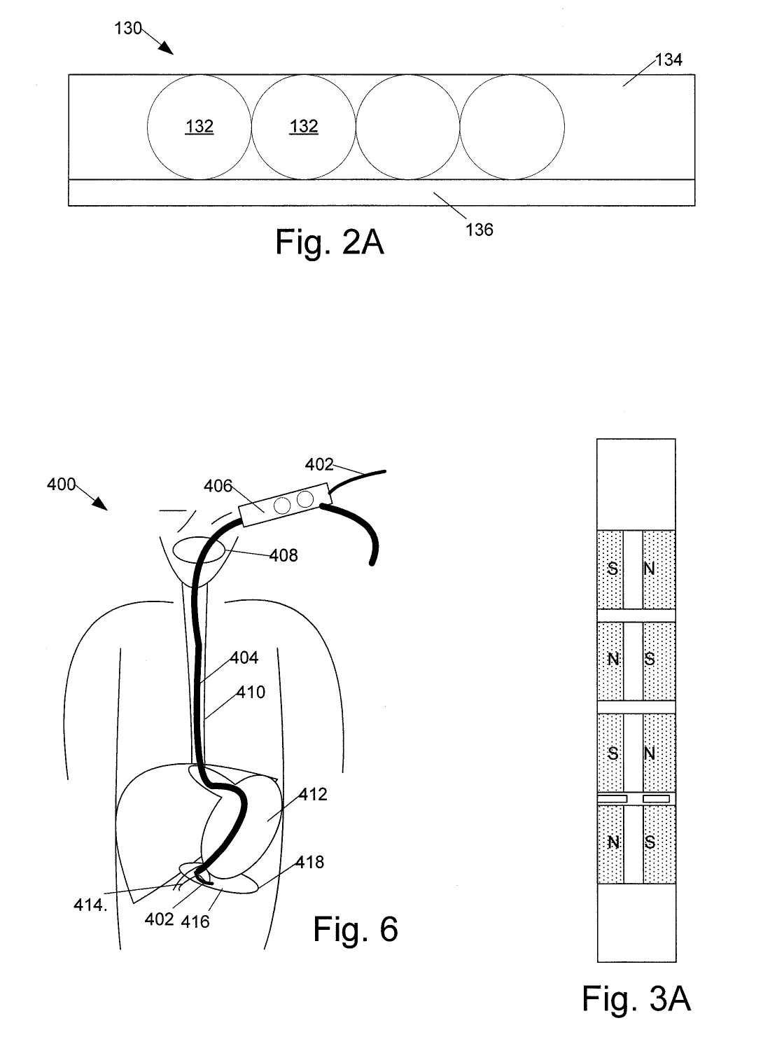

The magnetic portion 120 in an embodiment includes multiple segments of ferromagnetic or other magnetically permeable material 122 separated by spacers 124, the segments of magnetic material 122 may be ferrite beads (such as are commonly used for radiofrequency noise reduction) that are capable of concentrating magnetic flux and have a lumen 126 through them. The beads or segments are positioned in a sequence along the catheter. In an alternative embodiment, magnetic portion 130 (FIG. 2A) includes multiple segments, each segment formed as a ball 132 of ferromagnetic or other magnetically permeable material, such as soft iron or steel, that fill a first lumen 134 of the catheter. The balls are positioned in a sequence along the catheter. In embodiments where a first lumen 134 of the catheter is filled and obstructed by balls 132, a second lumen 136 may be provided for communicating with balloon 104, in the manner of a standard Foley catheter that has a first lumen for urine and a second lumen for a balloon. In all embodiments, an overall magnetic response along the magnetic portion is nonuniform, such that magnetic field gradients will form at multiple locations along the magnetic portion, not just at the ends, when an external static magnetic field is applied to the magnetic portion.

In an alternative embodiment 150 (FIG. 3), the magnetic portion has multiple cylindrical, ellipsoidal or spherical permanent magnets positioned in a sequence along the catheter within lumen 151, some of the magnets 152 are oriented with their poles in a first orientation, these magnets are alternated with magnets 154 having their poles oriented in a second, opposite, orientation such that a magnetic field gradient exists at multiple location along the catheter. The magnets 152, 154 are in most embodiments separated by spacers 156, although in some embodiments spacers 156 are omitted. The magnets may be arranged with alternating poles parallel to the long axis of the catheter as illustrated in FIG. 3A. If hollow cylindrical magnets having lateral poles, the magnets may be arranged with alternating poles perpendicular to the long axis of the catheter as illustrated in FIG. 3B.

Applicants note that some flexible polymer magnetic materials, such as those commonly used for refrigerator magnets, are magnetized with a sequence of alternating stripes where a first stripe of several millimeters width of the magnetic has magnetic poles oriented in a first direction, and an adjacent stripe of the magnetic material has magnetic poles oriented in a second direction; these stripes repeat in alternating orientation along a length of the material. In embodiments using such flexible polymer-based magnetic materials, the first and second direction are in some embodiments parallel to a long axis of the catheter, and in other embodiments are perpendicular to a long axis of the catheter.

For purposes of this document, a nonuniform permanent-magnet magnetic portion includes either flexible polymer magnetic materials with magnetization in alternating stripes, or multiple discrete magnets with alternating poles, and may have stripes or magnets oriented with poles either parallel to, or perpendicular to, the long axis of the catheter.

Use of the magnetic catheter of FIG. 1 is best understood by reference to the system diagram of FIG. 4 and the flowchart of FIG. 5.

After a male patient has been subjected to appropriate diagnostic procedures, including pre-operative imaging, and prostatic thermotherapy has been prescribed, the thermotherapy treatment method 200 begins with magnetic catheter 100, 168 of FIG. 1 being inserted 202 through the patient's penile urethra 161 in a manner similar to that of inserting a Foley catheter such that the balloon portion 104, 160, is located within his urinary bladder 162. In embodiments having a balloon 104, 160, the balloon may be inflated by injection of an aqueous solution from a syringe 164 through a lumen 166 of catheter 168; once balloon 104, 160 is inflated the lumen 166 may be sealed with a clamp or valve, or the syringe 164 clamped, to keep it inflated. The catheter is configured such that lumen 166, 136, or 126 extends from balloon 104 through the catheter to a proximal end, a distance from proximal to distal end being sufficient that it can extend through at least an average male human patient's urethra from tip of the penis and into the patient's urinary bladder with syringe 164 external to the urethra and balloon 104, 160 within the urinary bladder.

The magnetic portion 170 of catheter 168 is then positioned 206 such that the magnetic portion 106, 170 is within urethra of the prostate 172; in embodiments having a balloon 104, 160, this is accomplished by inserting the catheter fully so that the balloon is in the bladder 162, and inflating 204 the balloon, then gently withdrawing the catheter until the balloon seats at the point in the patient's bladder where the urethra exits the bladder. In other embodiments, visualization by fluoroscopy or transrectal ultrasound may be used to position the catheter.

Once the catheter is in position, in embodiments having a nonuniform magnetic portion formed of ferromagnetic or other magnetically permeable material, an external static magnetic field is applied 208 by energizing a coil 174 external to the patient with a direct current (DC) from power supply 176 to provide multiple magnetic field gradients along the prostate 172. In an alternative embodiment, a large external permanent magnet, or another large magnet such as an MRI machine main magnet, is applied to provide the static field. In embodiments using permanent magnets 152, 154, no external static magnetic field need be applied as the magnets of the magnetic portion directly provides multiple magnetic field gradients within the prostate.

Magnetic nanoparticles 178, in suspension in a carrier liquid in a second syringe 180 or other suitable pump are then injected 210 in such a way that at least a portion of nanoparticles 178 will follow the inferior vesicular artery 182 into prostate 172. In an embodiment, this is done by injecting nanoparticles 178 through a catheter 184 threaded through other arteries 186 to a point where its mouth is in or near inferior vesicular artery 182; in other embodiments it is performed by intravenous injection. A time is provided to allow magnetic nanoparticles 178 to be captured or trapped 212 by the nonuniform magnetic field within the prostate. In an alternative embodiment, prior to injecting the nanoparticles into or near the mouth of the inferior vesicular artery 182, a balloon 187 is inserted 209 into, and inflated within, the subject's rectum 189. Balloon 187 tends to compress blood vessels of the rectum, retarding flow of magnetic nanoparticle to the rectum and thereby helping to protect the heat-sensitive rectal lining from unintended thermal damage during thermotherapy of the prostate by reducing nanoparticle concentration in the rectal wall.

In an alternative embodiment, the magnetic nanoparticles are directly injected 210 into the prostate.

After a sufficient concentration of magnetic nanoparticles has reached the prostate, the catheter balloon 160, if present, is deflated, and the magnetic catheter 100, 168, is withdrawn 214 and any static magnetic field removed by turning off power supply 176 or removing any external permanent magnet. In an alternative embodiment where the static magnetic field is applied to the patient by inserting the patient into the field provided by the usually-toroidal main magnet of an underutilized magnetic resonance imaging (MRI) machine, the static field is removed by withdrawing the patient from the magnetic field. MRI machine main magnets are available in a fairly wide range of field intensity, from permanent magnets of under two tenths of a tesla to superconducting magnets of over three tesla, and provide ample field to affect positioning of magnetic nanoparticles.

Once the magnetic nanoparticles are positioned within the prostate, any rectal balloon 187 may be deflated 216 and removed from the patient.

Since the rectum is near the prostate, in an alternative embodiment, instead of applying a static magnetic field from outside the subject, the static magnetic field is applied by inserting a magnet, which in an embodiment is contained within balloon 187, into the rectum.

In a particular embodiment, through using small gaps between magnets, a magnetic field gradient of 15 tesla/meter is provided. Experiment shows this is sufficient field to trap magnetic nanoparticles in small arteries.

The patient is then provide at least one, and in some embodiments of the method more than one, treatment session 215. For each treatment session, a fiberoptic thermometry catheter 188, or other temperature-sensing device, is threaded up the urethra into, and positioned 216 within, the prostate in a manner similar to the way the magnetic catheter was inserted 202 and positioned, 206, and coupled to a temperature gauge 190.

Thermotherapy is then performed by applying 218 an alternating (AC) magnetic field by applying an AC current through power supply 176 to coil 174, with coil 174 located near the patient's perineum or lower abdomen. AC current is applied until a desired temperature is observed on gauge 190, at which time coil 174 is de-energized. AC current may optionally be re-applied in several pulses to achieve a desired, nearly-constant temperature versus time during each session. Alternatively, the magnitude of the AC current may be varied continuously to achieve a desired, nearly-constant temperature during each session. At the end of a treatment session, the fiber-optic thermometry catheter 188 is withdrawn 220.

In an embodiment, the magnetic catheter is configured to produce peak gradients and peak trapping of magnetic nanoparticles at a small distance from the urethra, not at the urethra itself; this helps serve to protect the urethra from excessive heating. In a particular embodiment, a magnetic field gradient of 15 tesla per meter is achieved, and peak nanoparticle trapping occurs between 5 and 10 millimeters from the magnetic catheter.

In another embodiment, the magnetic field gradient is increased with time during infiltration of the magnetic nanoparticles; in an embodiment by increasing strength of the static magnetic field. In an alternative embodiment, this is accomplished by increasing the numbers of permanent magnets within the magnetic catheter, in an embodiment by sequentially withdrawing and re-inserting catheters with different numbers and types of magnetic inserts. In this embodiment, particles are trapped at a first distance near the urethra by magnetic fields at a first strength, and particles are trapped at a second distance, greater than the first distance, by magnetic fields at a second strength greater than the first strength. The heating pattern of nanoparticles within the prostate is during subsequent application of the AC magnetic field is determined by distribution of the nanoparticles through the prostate.

It should be noted that AC magnetic field heating of the nanoparticles within the prostate may reach greater temperatures than heating of the urothelium within the urethra because of the distribution of magnetic nanoparticles within the prostate. Greater heating of prostate tumor and stroma than of the urothelium is desirable because excessive heating of the urothelium can cause problems for the subject such as scarring that could potentially close the urethral passageway. In an embodiment, the urothelium is actively cooled to prevent excess heating by passing chilled fluid through the thermometry catheter while the AC magnetic field is being applied.

In a particular embodiment, the urothelium, or esophageal endothelium, is cooled by inserting a cooling and temperature sensing catheter 900 (FIG. 9) into the urethra, or the esophagus, or whatever body opening is adjacent to the magnetic-nanoparticle-infiltrated organ to be treated. The catheter has an inner tube having a lumen 902 and inner tube wall 904, an outer tube having a lumen 906 and outer tube wall 908, the outer tube wall being closed at the end 909 inserted into the body opening. Spacers 910 (FIGS. 9 and 10), having holes 912 to permit passage of fluid, are provided at intervals along the catheter to ensure the inner tube 904 remains approximately centered within the outer tube lumen 906. In operation, during application of the AC magnetic field to the magnetic nanoparticles, a chilled nontoxic coolant, such as water or saline solution, flows through the inner tube lumen 902 into the cooling catheter, and allowed to return and exit through the outer lumen, thereby limiting temperature extremes at the urothelium or esophageal endothelium.

In a particular embodiment, one or more temperature sensors 914, which may be thermistors or other electronic temperature sensors known in the art of electronics, are embedded in the outer tube wall 908, and coupled through flexible leads 916 threaded through holes 912 in spacers 910 to temperature monitoring and display electronics (not shown).

Finally, post-treatment imaging may be performed to determine 222 any effect of the treatment, and whether additional treatment is necessary.

In an alternative embodiment 400 (FIG. 6), a catheter 100, 130, 402 is threaded through a catheter channel (not shown for simplicity) in an endoscope operating handle 406 and endoscope 404. Endoscope 404 is itself threaded through a patient's mouth 408, esophagus 410, stomach 412, and pyloris into a proximal portion of the patient's duodenum 414 adjacent an opening of one of the patient's pancreatic ducts 416. In a particular embodiment the catheter 402 lacks balloon 104 and balloon lumen 136, but has a nonuniform magnetic portion as previously described located at its distal end. The distal end of catheter 402, bearing the magnetic portion, is then threaded under visual observation through the endoscope into the pancreatic duct 416 within pancreas 418. Once the catheter is positioned within the pancreatic duct, the steps of applying a static magnetic field 208, injecting nanoparticles 210, and allowing nanoparticles to accumulate 212 are performed as previously described with reference to the prostate, except that the particles are injected in a manner that they will enter an artery perfusing the pancreas, and the injected nanoparticles are allowed to accumulate in the pancreas. In an alternate embodiment, the nanoparticles are injected in a vein. The static field, if any, is then removed, a fiberoptic thermometry catheter is optionally threaded into the pancreatic duct, and one or more sessions of an AC magnetic field is applied transcutaneously to the nanoparticles in the pancreas. In embodiments, a temperature of the pancreas is monitored with a fiberoptic thermometry catheter is threaded into the pancreatic duct while the AC magnetic field is applied to the pancreas to permit control of heating of the pancreas. This alternative embodiment is useful for treating some disorders of the pancreas.

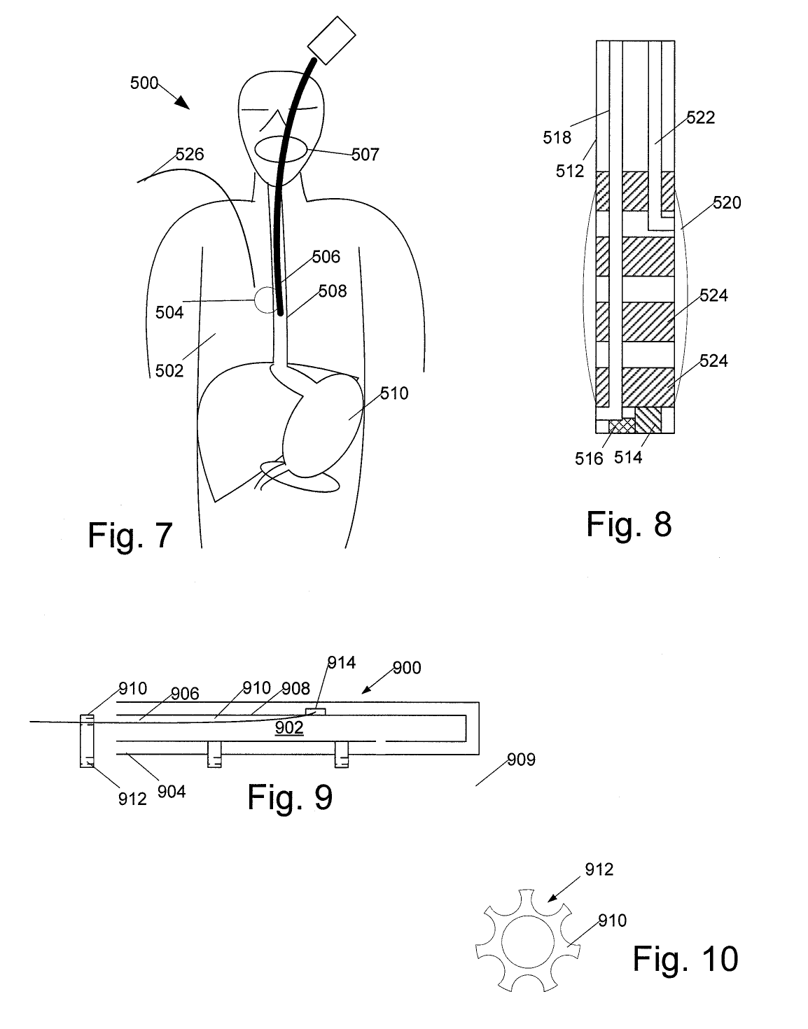

In an alternative embodiment 500, illustrated in FIG. 7, a subject 502 is treated using a magnetic catheter as previously described with reference to FIGS. 1-3, without balloon 104. In an alternative embodiment, subject 502 is treated using a magnetic endoscope as illustrated in FIG. 8. In both embodiments, the magnetic catheter or endoscope 506 is passed through the subject's mouth 507 and positioned in a subject's esophagus 508 or stomach 510 near tissue to be treated, such as a tumor 504. In embodiments using the endoscope of FIG. 8, positioning of endoscope 506, 512 may be assisted by observation using a miniature video camera 514 under illumination of an LED 516. Air may be admitted or released through an observation-enhancement gas lumen 518, and steering wires (not shown) as known in the art of endoscopes may be provided. Once positioned, in some particular embodiments an optional balloon 520 may be inflated by gas passed through an inflation lumen 522 to apply pressure on, and retard blood flow and accumulation of magnetic nanoparticles in, esophageal lining. The magnets, or magnetically permeable inserts in cooperation with an external magnet (not shown), 524 of the catheter or endoscope then serve as previously described to cause magnetic gradients near the tissue to be treated 504. Magnetic nanoparticles are then injected through a needle or catheter 526 into, or into a vessel that perfuses, the tissue to be treated 504. A similar embodiment may also be used to treat tissue near trachea or bronchi, however an open lumen through the catheter or endoscope may be desirable to ensure adequate ventilation of subject 502.

Once the nanoparticles are injected, and have accumulated in the tissue to be treated, the magnetic catheter or endoscope is removed, a temperature sensing catheter or endoscope is positioned to monitor treatment, and an AC magnetic field is applied to cause heating of the tissue to be treated. Since esophageal lining is easily damaged, and healing after surgery on the esophagus is often slow and problematic, the temperature sensing catheter or endoscope of a particular embodiment is water-cooled to prevent undue heat damage to the lining.

In some alternative embodiments, magnetic nanoparticles are injected into selected vessels, which are then heated by applying the AC magnetic fields described herein. The heat can destroy the vessels, which in turn deprives nearby tumor of blood flow and destroys the tumor. In a particular embodiment, the magnetic nanoparticles are injected in large volumes into either arteries upstream of the vessels to be treated. or into systemic veins, and are trapped in blood vessels by the magnetic field gradients near the magnetic catheter herein described, these particles effectively aggregate and obstruct blood flow in those vessels, depriving nearby tumor or benign prostate hyperplasia tissue of blood and destroying tumor or hyperplastic tissue, even without heating. In this particular embodiment, particles not aggregated by magnetic gradients near the magnetic catheter circulate unimpeded until trapped by the magnetic field gradients near the magnetic catheter, or are sequestered or excreted by the body. For this reason, injection of nanoparticles can be made into a vein, or into an artery one or more branches upstream of the artery supplying the prostatic tissue to be destroyed by arterial embolism. It is not necessary to navigate the injection apparatus under fluoroscopic or other guidance past a succession of arterial branches into successively smaller arteries that may be brittle due to atherosclerosis in order to perform the injection immediately upstream of the tissue to be destroyed. Arterial embolism by aggregation of the magnetic nanoparticles occurs only in close proximity to the catheter inserted within the urethra, not in other arteries through which the injected magnetic nanoparticles may flow after injection.

In an alternative embodiment, the magnetic nanoparticles are embedded in a water-soluble matrix that is substantially loses water solubility when heated above a transition or polymerization temperature by exposure to the AC magnetic field.

### Combinations

The features herein described may be present in various combinations in a particular system. For example, and not by limitation, the urethral catheter, endoscope-threaded, endoscopic, and esophageal implementations above described may have nonuniform magnetic portions of either the spaced-magnetically-permeable segment type or of the nonuniform permanent magnet type. Particular embodiments may include:

A magnetic catheter designated A including a flexible, shaft, a nonuniform magnetic portion comprising nonuniform magnetic elements selected from a nonuniform permanent magnet portion and a nonuniform magnetically permeable portion.

A magnetic catheter designated AA including the catheter designated A wherein the flexible shaft includes a flexible tube having at least a first lumen, the first lumen communicating with a balloon, the balloon located near a distal end of the flexible tube; wherein the nonuniform magnetic portion is located near, but proximal to, the balloon; and wherein the catheters is adapted to having the distal end inserted through a penile urethra into a urinary bladder of a male patient, the first lumen extending from the distal end to a proximal end and configured such that injection of fluid into a proximal end of the first lumen can inflate the balloon.

A magnetic catheter designated AB including the catheter designated A, the catheter being adapted to be inserted through a lumen of an endoscope by having sufficient length, flexibility and small diameter to fit through the lumen of the endoscope.

An assembly including the magnetic catheter designated ABA including the catheter designated AB, further combined with an endoscope, the endoscope adapted for usage in the upper gastrointestinal tract.

A magnetic catheter designated AC including the catheter designated A, wherein the flexible shaft comprises a flexible tube having at least a first lumen, the first lumen of the flexible shaft communicating with a balloon, the balloon located around the a nonuniform magnetic portion; and wherein the tube is adapted to having the distal end inserted into the esophagus or anus of a patient.

A magnetic catheter designated AD including the magnetic catheter designated A, AA, AB, ABA, or AC wherein the nonuniform magnetic portion comprises at least one magnetically permeable portion.

A magnetic catheter designated AE including the magnetic catheter designated AD wherein the nonuniform magnetic portion comprises a plurality of magnetically permeable portions in a sequence along a length of the magnetic portion.

A magnetic catheter designated AF including the magnetic catheter designated AE wherein the magnetically permeable portions are separated by spacers.

A magnetic catheter designated AG including the magnetic catheter designated A, AA, AB, ABA, or AC wherein the nonuniform magnetic portion comprises a nonuniform permanent-magnet magnetic portion.

A magnetic catheter designated AH including the magnetic catheter designated AG wherein the nonuniform magnetic portion comprises a plurality of permanent magnets disposed in a sequence along a length of the magnetic portion.

A system designated B including a magnetic catheter having a flexible shaft, and a nonuniform magnetic portion comprising nonuniform magnetic elements selected from a nonuniform permanent magnet portion and a nonuniform magnetically permeable portion; apparatus for administering magnetic nanoparticles to a subject; and apparatus for applying an alternating magnetic field to the subject.

A system designated BA including the system designated B wherein the flexible shaft is a flexible tube having at least a first lumen, the first lumen communicating with a balloon, the balloon located near a distal end of the flexible tube; wherein the nonuniform magnetic portion is located near, but proximal to, the balloon; and where the tube is adapted to having the distal end inserted through a penile urethra into a urinary bladder of a male patient, the first lumen extending from the distal end to a proximal end and configured such that injection of fluid into a proximal end of the first lumen can inflate the balloon.

A system designated BB including the system designated B, further including an endoscope adapted for use in a gastrointestinal tract, and wherein the magnetic catheter is adapted to be inserted through a lumen of the endoscope by having sufficient length, flexibility and small diameter to fit through said endoscope.

A system designated BC including the system designated B, wherein the flexible shaft has a flexible tube having at least a first lumen, the first lumen of the flexible shaft communicating with a balloon, the balloon located around the a nonuniform magnetic portion; and wherein the tube is adapted to having the distal end inserted into the esophagus or anus of a patient.

A system designated BD including the system designated B, BA, BB or BC and further including a magnet configured to apply a DC magnetic field to a subject, and wherein the nonuniform magnetic portion comprises at least one magnetically permeable portion.

A system designated BE including the system designated BD wherein the nonuniform magnetic portion comprises a plurality of magnetically permeable portions in a sequence along a length of the magnetic portion.

A system designated BF including the system designated BE wherein the magnetically permeable portions are separated by spacers

A system designated BG including the system designated B, BA, BB, or BC wherein the nonuniform magnetic portion comprises a nonuniform permanent-magnet magnetic portion.

A system designated BH including the system designated B, BA, BB, or BC, wherein the nonuniform magnetic portion comprises a plurality of permanent magnets disposed in a sequence along a length of the magnetic portion.

A method designated C of treating an organ of a patient includes applying a nonuniform magnetic field to the organ by a method including inserting a catheter having a nonuniform magnetic portion into a lumen of a duct or vessel of the patient located within the organ; injecting magnetic nanoparticles into the patient; allowing the magnetic nanoparticles to concentrate in the nonuniform magnetic field within the organ; removing the catheter; and applying a time-varying magnetic field to the magnetic nanoparticles.

A method designated CA including the method designated C where the duct is a urethra and the organ is a prostate, and the catheter has a balloon portion, and farther comprising inflating the balloon portion to guide positioning of the nonuniform magnetic portion into the prostate.

A method designated CB, CF, or CG including the method designated C or CA wherein the nonuniform magnetic portion comprises a plurality of permanent magnets.

A method designated CC, CF, or CG including the method designated C or CA wherein the nonuniform magnetic portion comprises a plurality of magnetically permeable objects, and wherein applying a nonuniform magnetic field to the prostate further comprises applying a DC (or static) magnetic field from a magnet external to the patient.

A method designated CD including the method designated C, CA, CB, or CC, wherein the organ is the prostate and further comprising monitoring a temperature of the prostate.

A method designated CE including the method designated C, CA, CB, CC, or CD, wherein the magnetic portion is positioned to avoid undue accumulation of nanoparticles in the internal and external urinary sphincters.

A method designated CF including the method designated C, wherein the organ is a pancreas.

A method designated CG including the method designated CF and further including threading the catheter through a lumen of an endoscope into a pancreatic duct.

A system designated D and adapted for treatment of the prostate has a magnetic catheter with a nonuniform magnetic portion located near a distal end of the catheter, the catheter adapted to be inserted through a penile urethra into a urinary bladder of a male patient; a suspension of magnetic nanoparticles contained within an apparatus adapted to inject the nanoparticles into the patient; and apparatus adapted to provide an alternating magnetic field to a prostate of the patient.

A system designated DA including the system designated D wherein the nonuniform magnetic portion of the catheter comprises at least one magnetically permeable portion, and the system further comprises a magnet configured to provide a static magnetic field to the prostate of the patient.

A system designated DB including the system designated D wherein the nonuniform magnetic portion of the catheter comprises a plurality of permanent magnets.

Any of the system or methods above described wherein the magnetic nanoparticles have average diameter between ten and one thousand nanometers.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1-4. (canceled)

5. A magnetic catheter comprising:
a flexible shaft;
a nonuniform magnetic portion comprising nonuniform magnetic elements selected from a nonuniform permanent magnet portion and a nonuniform magnetically permeable portion;
wherein the flexible shaft comprises a flexible tube having at least a first lumen, the first lumen of the flexible shaft communicating with a balloon, the balloon located around the a nonuniform magnetic portion; and
wherein the tube is adapted to having the distal end inserted into the esophagus of a patient.

6. The magnetic catheter of claim 5 wherein the nonuniform magnetic portion comprises at least one magnetically permeable portion.

7. The magnetic catheter of claim 6 wherein the nonuniform magnetic portion comprises a plurality of magnetically permeable portions in a sequence along a length of the magnetic portion.

8. The magnetic catheter of claim 7 wherein the magnetically permeable portions are separated by spacers.

9. The magnetic catheter of claim 5 wherein the nonuniform magnetic portion comprises a nonuniform permanent-magnet magnetic portion.

10. The magnetic catheter of claim 9 wherein the nonuniform magnetic portion comprises a plurality of permanent magnets disposed in a sequence along a length of the magnetic portion.

11-13. (canceled)

14. A system comprising:
a magnetic catheter comprising:
a flexible shaft, and
a nonuniform magnetic portion comprising nonuniform magnetic elements selected from a nonuniform permanent magnet portion and a nonuniform magnetically permeable portion;
apparatus for administering magnetic nanoparticles to a subject; and
apparatus for applying an alternating magnetic field to the subject
wherein the flexible shaft comprises a flexible tube having at least a first lumen, the first lumen of the flexible shaft communicating with a balloon, the balloon located around the a nonuniform magnetic portion; and
wherein the tube is adapted to having the distal end inserted into the esophagus of a patient.

15. The system of claim 14 further comprising a magnet configured to apply a static magnetic field to a subject, and wherein the nonuniform magnetic portion comprises at least one magnetically permeable portion.

16. The magnetic catheter of claim 15 wherein the nonuniform magnetic portion comprises a plurality of magnetically permeable portions in a sequence along a length of the magnetic portion.

17. The magnetic catheter of claim 16 wherein the magnetically permeable portions are separated by spacers.

18. A system comprising:
a magnetic catheter comprising:
a flexible shaft, and
a nonuniform magnetic portion comprising nonuniform magnetic elements selected from a nonuniform permanent magnet portion and a nonuniform magnetically permeable portion;
apparatus for administering magnetic nanoparticles to a subject; and
apparatus for applying an alternating magnetic field to the subject
wherein the nonuniform magnetic portion comprises a nonuniform permanent-magnet magnetic portion.

19. The magnetic catheter of claim 18 wherein the nonuniform magnetic portion comprises a plurality of permanent magnets disposed in a sequence along a length of the magnetic portion.

20. A method of treating an organ of a patient comprising:
applying a nonuniform magnetic field to the organ by a method including inserting a catheter having a nonuniform magnetic portion into a lumen of a duct or vessel of the patient located within the organ;
injecting magnetic nanoparticles into the patient;
allowing the magnetic nanoparticles to concentrate in the nonuniform magnetic field within the organ;
removing the catheter; and
applying a time-varying magnetic field to the magnetic nanoparticles.

21. The method of claim 20 wherein the duct is a urethra and the organ is a prostate, and the catheter has a balloon portion, and further comprising inflating the balloon portion to guide positioning of the nonuniform magnetic portion into the prostate.

22. The method of claim 21 wherein the nonuniform magnetic portion comprises a plurality of permanent magnets.

23. The method of claim 21 wherein the nonuniform magnetic portion comprises a plurality of magnetically permeable objects, and wherein applying a nonuniform magnetic field to the prostate further comprises applying a DC magnetic field from a magnet external to the patient.

24. The method of claim 21 further comprising monitoring a temperature of the prostate.

25. The method of claim 24 wherein the magnetic portion is positioned to avoid undue accumulation of nanoparticles in the internal and external urinary sphincters.

26. The method of claim 20 wherein the organ is a pancreas.

27. The method of claim 26 further comprising threading the catheter through a lumen of an endoscope into a pancreatic duct.

28. The method of claim 27 wherein the nonuniform magnetic portion comprises a plurality of permanent magnets.

29. The method of claim 27 wherein the nonuniform magnetic portion comprises a plurality of magnetically permeable objects, and wherein applying a nonuniform magnetic field to the prostate further comprises applying a DC magnetic field from a magnet external to the patient.

30. (canceled)

31. A system adapted for treatment of the prostate comprising:
a magnetic catheter comprising a nonuniform magnetic portion located near a distal end of the catheter, the catheter adapted to be inserted through a penile urethra into a urinary bladder of a male patient;
a suspension of magnetic nanoparticles contained within an apparatus adapted to inject the nanoparticles into the patient; and
apparatus for providing an alternating magnetic field to a prostate of the patient
wherein the nonuniform magnetic portion of the catheter comprises at least one magnetically permeable portion, and the system further comprises a magnet configured to provide a static magnetic field to the prostate of the patient.

32-33. (canceled)

34. The system of claim 14 wherein the magnetic nanoparticles have average diameter between ten and one thousand nanometers.

35. A method of treating an organ of a patient comprising:
applying a nonuniform magnetic field to the organ by a method including inserting a catheter having a nonuniform magnetic portion into a lumen of a duct or vessel of the patient located within the organ;
injecting magnetic nanoparticles into the patient; and
allowing the magnetic nanoparticles to concentrate in the nonuniform magnetic field within the organ in sufficient density to obstruct blood flow in at least one artery of the patient.

36. A system adapted for treatment of the prostate comprising:
a magnetic catheter comprising a nonuniform magnetic portion located near a distal end of the catheter, the catheter adapted to be inserted through a penile urethra into a urinary bladder of a male patient; and
a suspension of magnetic nanoparticles contained within an apparatus adapted to inject the nanoparticles into the patient;
wherein the system is capable of applying magnetic field gradients sufficiently intense to aggregate magnetic nanoparticles and obstruct an artery in a prostate of the patient.
